(19) United States
(12) Patent Application Publication
(10) Pub. No.: US 2005/0020994 A1
(43) Pub. Date: Jan. 27, 2005

(54) Process for applying portions of material to a moving web

(76) Inventors: Tony C. Ng, East Brunswick, NJ (US); Steven H. White, Somerset, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/827,571

(22) Filed: Oct. 6, 2004

Related U.S. Application Data
(62) Division of application No. 10/284,622, filed on Oct. 31, 2002.

(51) Int. Cl. A61F 13/15; A61F 13/20

(52) U.S. Cl. 604/389

(57) ABSTRACT

An absorbent article comprising an absorbent body enclosed between a liquid-permeable liner sheet layer and a liquid-impermeable backsheet layer is disclosed. The article has a longitudinal axis, a relatively wide anterior end and an opposite posterior end connected by side margins to define a generally triangular shape. The side margins substantially continuously converge to the posterior end, and at least one tab extends from each side margin. There is at least one attachment element aligned oblique to the longitudinal axis of the article that is associated with each tab. A method of placing an attachment element onto an absorbent article at an angle that is oblique to a machine direction is also disclosed, along with a product formed by the method. A first attachment element is separated from a first supply of attachment material that is aligned in a machine direction and is rotated from the machine direction to a predetermined angle that is oblique to the machine direction. This attachment element is indexed to an absorbent article moving in the machine direction and is applied to the absorbent article. A second attachment element may be similarly applied to the absorbent article, and it may be aligned opposite the first attachment element. An apparatus for placing at least one attachment element onto an absorbent product at an angle that is oblique to a machine direction is also disclosed.

### FIELD OF THE INVENTION

This invention relates to a method and apparatus for laminating a selected substrate material at an oblique angle to a moving web and a product made therefrom. More particularly, the present invention relates to a method and apparatus for adhering separate portions of material to a moving web in the construction of an absorbent article, such as a disposable diaper or sanitary napkin.

### BACKGROUND OF THE INVENTION

Disposable absorbent articles, such as sanitary napkins, pantiliners, diapers, training pants, incontinence garments and the like, have typically employed adhesive tape to secure the absorbent article to the wearer's undergarments or about the waist of the wearer. As designs have become more complex, it has become desirable to have adhesive tabs that are oriented at an oblique angle to the product. This angle permits more economical use and more appropriate orientation of materials.

U.S. Pat. No. 5,713,886 to Sturino describes a sanitary napkin or pantiliner, especially for use with G-string and thong type undergarments. The product has (1) an elongated, relatively narrow, first end portion having a generally uniform width and (2) a generally triangular second end portion having a relatively wide second end extending therefrom. This product is relatively complex, and it would require significant manipulation by the user in order to apply it to her undergarments. It would also be relatively costly to manufacture with significant waste generated. Finally, the reference fails to disclose how to manufacture its complex product in a commercially viable manner.

U.S. Pat. No. 2,289,336 to A. Bamford describes a method and apparatus for applying windows of transparent material to a moving base material. The apparatus employs a rotary suction carrier that moves portions of sheet material into an applying zone at a constant rate of continuous travel. A mechanism displaces individual suction elements outwardly and substantially radially of the suction carrier to apply individual portions of sheet material to the base material.

Another technique for applying window portions to a moving web is described in U.S. Pat. No. 4,642,085 to Helm. An apparatus described by Helm feeds a web of material to an adhesive applying mechanism. A vacuum conveyor transports the web from the adhesive applying mechanism to a severing mechanism which cuts successive window portions from a preselected length of the web in spaced relation to a portion of the web engaged on the surface of a vacuum roll. The vacuum roll then applies the portions to a moving base sheet layer.

Other techniques for applying portions to a moving web are described in U.S. Pat. No. 2,958,437 to Mengis; U.S. Pat. No. 4,061,527 to Traise; U.S. Pat. No. 3,933,564 to Jensen; U.S. Pat. No. 4,475,969 to Reed; and U.S. Pat. No. 4,795,510 to Wittrock et al.

Conventional techniques, such as those described above, have not been able to provide a method and apparatus for laminating a selected substrate material at an oblique angle to a moving web and a product made therefrom. As a result, conventional techniques have not been adequate for tasks such as applying adhesive portions to angled regions of moving webs, e.g., in the manufacture of absorbent articles. The techniques have generated excessive waste by covering more area than necessary.

### BRIEF DESCRIPTION OF THE INVENTION

It is an object of the present invention to provide a disposable article having an attachment element aligned oblique to the longitudinal axis of the article.

It is another object of the present invention to provide a method of placing an attachment element onto an absorbent article at an angle that is oblique to a machine direction of the process equipment.

In accordance with one embodiment of the present invention, an absorbent article comprising an absorbent body enclosed between a liquid-permeable liner sheet layer and a liquid-impermeable backsheet layer is disclosed. The article has a longitudinal axis, a relatively wide anterior end and an opposite posterior end connected by side margins to define a generally triangular shape. The side margins substantially continuously converge to the posterior end, and at least one tab extends from each side margin. There is at least one attachment element aligned oblique to the longitudinal axis of the article that is associated with each tab.

In accordance with another embodiment of the invention a method of placing an attachment element onto an absorbent article at an angle that is oblique to a machine direction is disclosed. A first attachment element is separated from a first supply of attachment material that is aligned in a machine direction and is rotated from the machine direction to a predetermined angle that is oblique to the machine direction. This attachment element is indexed to an absorbent article moving in the machine direction and is applied to the absorbent article. A second attachment element may be similarly applied to the absorbent article, and it may be aligned opposite the first attachment element.

Another embodiment of the invention relates to an absorbent body enclosed between a liquid-permeable liner sheet layer and a liquid-impermeable backsheet layer. The article has a longitudinal axis, a relatively wide anterior end and a posterior end connected by side margins to define a generally triangular shape. There is at least one tab extending from each side margin, and a process largely as described above forms the article.

In accordance with yet another embodiment of the invention, an apparatus for placing at least one attachment element onto an absorbent product at an angle that is oblique to a machine direction is disclosed. The apparatus includes a frame, a fixed cylindrical camshaft mounted on the frame having at least one fixed cam track, a revolving cylindrical anvil sleeve mounted on the fixed camshaft, and at least one rotatable disk disposed at the surface of the anvil sleeve. The rotatable disk has a cam follower arranged and configured to engage the at least one fixed cam track, and it is capable of placing the at least one attachment element onto the absorbent product. The frame, camshaft, and anvil sleeve define a machine direction and the at least one rotatable disk is rotated to a predetermined angle that is oblique to the machine direction when the anvil sleeve is in position to place the at least one attachment element onto the absorbent product.

### BRIEF DESCRIPTION OF THE DRAWINGS

The present invention will be more fully understood and further advantages will become apparent when reference is made to the following detailed description of the invention and the drawings, in which:

FIG. 1 representatively shows the backsheet side of a sanitary napkin produced with the present invention;

FIG. 2 representatively shows an isometric view of the apparatus of the invention wherein the coated substrate is introduced at an oblique angle to the direction of travel of the web material;

FIG. 3 representatively shows an exploded isometric view of the anvil roller and camshaft of the apparatus of FIG. 2;

FIG. 4 representatively shows a cross-section along line 44 of the roller section of the apparatus of FIG. 2;

FIG. 5 representatively shows a cross-section along line 5-5 of the anvil roller and knife roller of the apparatus of FIG. 2; and

FIG. 6 representatively shows a view of the surface of the anvil roller of the apparatus of FIG. 2.

### DETAILED DESCRIPTION OF THE PREFERRED EMBODIMENTS

The method and apparatus of the present invention are particularly suitable for adhesively securing portions of material to a moving web in the construction of an absorbent article, such as a sanitary napkin, pantiliner, disposable diaper, incontinence garment, training pant or the like.

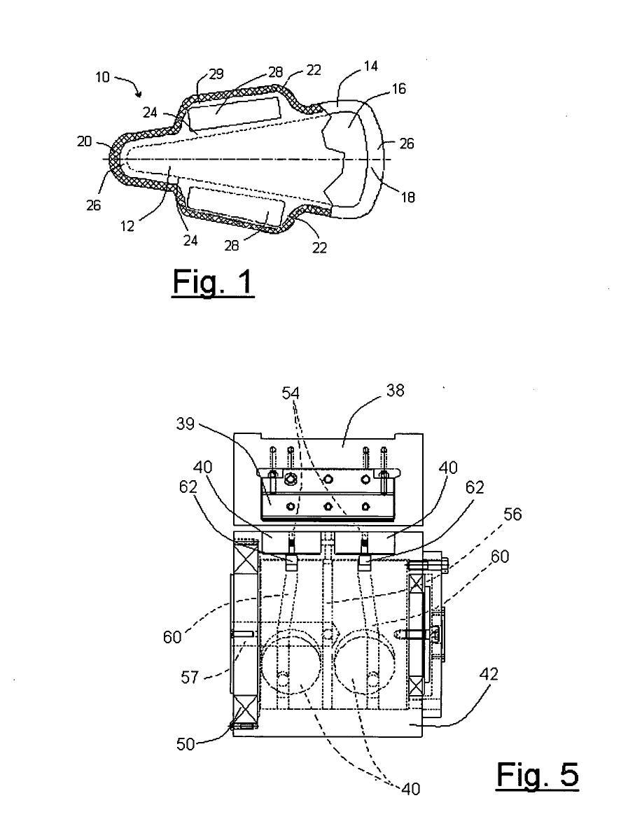

With reference to FIG. 1, an absorbent article, such as sanitary napkin 10, generally comprises a backsheet layer 12, a substantially liquid-permeable liner sheet layer 14, and an absorbent body 16 sandwiched between the backsheet and liner layers. Napkin 10 has a longitudinal axis, a relatively wide anterior end 18, and it tapers to a posterior end 20. Napkin 10 thus has a generally triangular planform with tabs 22 extending oppositely along the lateral, cross-wise direction of the napkin. In the shown embodiment, backsheet 12 and liner sheet 14 are essentially coterminous and extend out past the edges of absorbent body 16 to form side margins 24 and end margins 26. The two tabs 22 extending from the side margins 24 include attachment elements that are useful to fasten the napkin on the wearer's undergarment. Numerous materials useful as attachment elements are known in the art and include pressure sensitive adhesives, cohesive-adhesives, frictional coatings, straps, belts, snaps, and the like. In the illustrated embodiment, the attachment elements comprise adhesive portions 28.

When such absorbent articles 10 are in use, the tabs 22 are folded over the edges of the wearer's undergarment. They thus may more firmly secure the article to the undergarment, stabilize the article within the undergarment, provide an increased absorptive area for bodily exudates, and help prevent the undergarment from becoming soiled in part by protecting the side edges of the wearer's undergarment. Tabs of the present invention may comprise an integral extension of the materials (or some of the materials) of which the main body of the article is formed. As an example, the tabs may comprise extensions of the coversheet layer material and the backsheet layer material that are joined together, e.g., about the periphery of the tab. They may also comprise tabs constructed from panels which themselves comprise integral extensions of the materials of which the main body of the article is formed, having been re-affixed to the underside of the main body (i.e. on the garment facing-surface); all as described in commonly assigned co-pending U.S. pat. app. Ser. No. 08/996,141, filed Dec. 22, 1997, herein incorporated by reference in its entirety. Alternatively, the tabs may comprise additional material added to the main body of the article after the main body's formation, typically secured to the garment-facing surface of the main body.

In the preferred embodiment shown in this figure, tabs 22 are centrally located centrally along the side margin. These tabs are spaced from the anterior and posterior ends. This permits the use of smaller, less obtrusive tabs for use with minimal undergarments, such as thong and g-string type undergarments. While the illustrated embodiment has two tabs, it is apparent that at least one tab is used, and the discussion above relating to two tabs is also relevant when fewer or more tabs are employed.

The backsheet layer 12 is typically composed of a polymer film, such as polypropylene, polyethylene, polyester, and blends thereof. The film may be oriented to provide increased strength, or it may be an unoriented polypropylene film having a nonglossy, matte finish. In addition, the film may be printed or embossed with decorations or writings, as desired. Alternatively, backsheet layer 12 may be composed of a nonwoven fibrous web, such as a spunbonded web, a meltblown web, a bonded-carded-web or the like, composed of natural or synthetic fibers or blends thereof. For example, where backsheet layer 12 is composed of a thin polymer film, the film thickness can be within the range of about 0.0025-0.0040 cm. In embodiments in which the web layer is composed of a nonwoven fibrous web, the fibrous web can have a basis weight within the range of about 25-70 g/m2.

The absorbent fibrous web may have a substantially uniform density as a result of compression by, e.g., smooth calender rollers, or it may have at least one portion of increased density as a result of an embossing process. If the web is embossed, higher levels of bulk may be maintained. Such structures are useful for some diapers and other bulkier absorbent products.

The liner sheet layer 14 may be a relatively low density, bulky, high-loft non-woven web material. It may be composed of homogeneous fibers, such as polyester or polypropylene or it may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof. An example is the non-woven cover layer of sanitary napkins sold by Johnson & Johnson Inc. of Montreal, Canada under the trademark Stayfree Ultra-Thin Cottony Dry Cover.

Bi-component fibers may be made up of a polyester core and a polyethylene sheath. The use of appropriate bi-component materials results in a fusible non-woven fabric. Examples of such fusible fabrics are described in U.S. Pat. No. 4,555,446 issued Nov. 50, 1985 to Mays, the disclosure of which is herein incorporated by reference. Using a fusible fabric increases the ease with which the cover layer may be mounted to the adjacent first absorbent layer and/or to the barrier layer.

The liner sheet layer 14 preferably has a relatively high degree of wettability, although the individual fibers comprising the cover may not be particularly hydrophilic. The cover material can also contain a great number of relatively large pores. This is because the liner sheet layer 14 is intended to take-up body fluid rapidly and to transport it away from the body and the point of deposition. The liner sheet layer 14 may be treated to allow fluid to pass through it readily. The liner sheet layer 14 may also function to transfer the fluid quickly to the other layers of the absorbent body 16.

Alternatively, the liner sheet layer 14 can also be made of polymer film having large pores. Because of such high porosity, the film accomplishes the function of quickly transferring body fluid to the inner layers of the absorbent system. Apertured co-extruded films such as described in U.S. Pat. No. 4,690,679, herein incorporated by reference, and available on sanitary napkins sold by Johnson & Johnson Inc. of Montreal, Canada could be useful as cover layers in the present invention.

The liner sheet layer 14 may be embossed to the absorbent body 16 in order to aid in promoting fluid transport by fusing the cover to the next layer. Such fusion may be effected locally, at a plurality of sites or over the entire contact surface of liner sheet layer 14 with absorbent body 16. Alternatively, the liner sheet layer 14 may be attached to the absorbent body 16 by other means such as by adhesive.

Adjacent to the liner sheet layer 14 on its inner side and preferably bonded to the liner sheet layer 14 is the absorbent body 16. The absorbent body 16 may be a uniform structure, or there may be different regions of the absorbent body 16 with different fluid management characteristics. These regions may be provided by a plurality of layers or by different locations about the generally planar absorbent body 16. The absorbent body 16 may be composed of cellulosic materials such as wood pulp, cotton, rayon, flax, jute, hemp, peat moss, and the like; polymeric materials such as polyesters, polyvinyl alcohols, polyolefins, polyamines, polyamides, polyacrylonitriles, SAP (superabsorbent polymers), hydrogels, and the like; or combinations of these materials. The materials may be in the form of fibers, foams, and particles or other discrete materials.

The absorbent body 16 can contain any superabsorbent polymer (SAP), which are well known in the art. For the purposes of the present invention, the term “superabsorbent polymer” (or “SAP”) refers to materials that are capable of absorbing and retaining at least about 10 times their weight in body fluids under a 0.5 pounds/in2 (psi) pressure. The superabsorbent polymer particles of the invention may be inorganic or organic cross-linked hydrophilic polymers, such as polyvinyl alcohols, polyethylene oxides, cross-linked starches, guar gum, xanthan gum, and the like. The particles may be in the form of a powder, grains, granules, or fibers.

The liner sheet layer 14 and the backsheet layer 12 are joined along their marginal portions so as to form an enclosure or flange seal that maintains the absorbent body 16 captive. The joint may be made by means of adhesives, heat bonding, ultrasonic bonding, radio frequency sealing, mechanical crimping, and the like and combinations thereof. The peripheral zone in which the layers are joined is shown in FIG. 1 by the reference numeral 29. The side margins 24 preferably begin at the side edges of absorbent body 16. These side margins 24 generally have a substantially straight inner edge, although an arcuate inner edge is possible. Thus, a straight line or a slight concave or convex curve may define this inner edge. Preferably, the inner edge of the side margins 24 does not have a significant break or change in its direction. Thus, the napkin 10 has a generally triangular plan or shape with side margins 24 that are aligned at an angle oblique to the longitudinal axis. The oblique alignment of the side margins 24 provides the tabs 22 with a similar angle. Thus, each attachment element 26 is also aligned at an angle oblique to the longitudinal axis.

The attachment element 26 includes a structure that defines an alignment of the attachment element. As shown in FIG. 1, a release liner covers an adhesive portion, and each of these two elements has a generally rectangular shape. The rectangular adhesive portion can be either a continuous area or defined by a pattern of adhesive elements. This rectangular shape has a longitudinal axis and a side edge that are aligned oblique to the absorbent article's longitudinal axis. Of course, other shapes or patterns will have an alignment, for example defined by a longitudinal axis of an elongate shape, and some complex shapes may have several directions of alignment defined by several major axes. Again, if a generally rectangular release liner protects these complex shapes, the release liner's longitudinal axis or side edge can define the alignment of the attachment element.

Conventional in-line techniques for applying attachment elements onto absorbent article substrates have not been completely satisfactory. One such conventional technique completely covers the angled region. This can be wasteful of the attachment element and, if the element is a pressure sensitive adhesive, of expensive release liners. It can also result in overspray or other sloppy applications of such an adhesive. This required added maintenance of production equipment. Another conventional technique applies numerous elements aligned either in the machine direction or cross-direction. This technique may not provide adequate coverage of the angled area if such alignment does not properly fit on the angled portion.

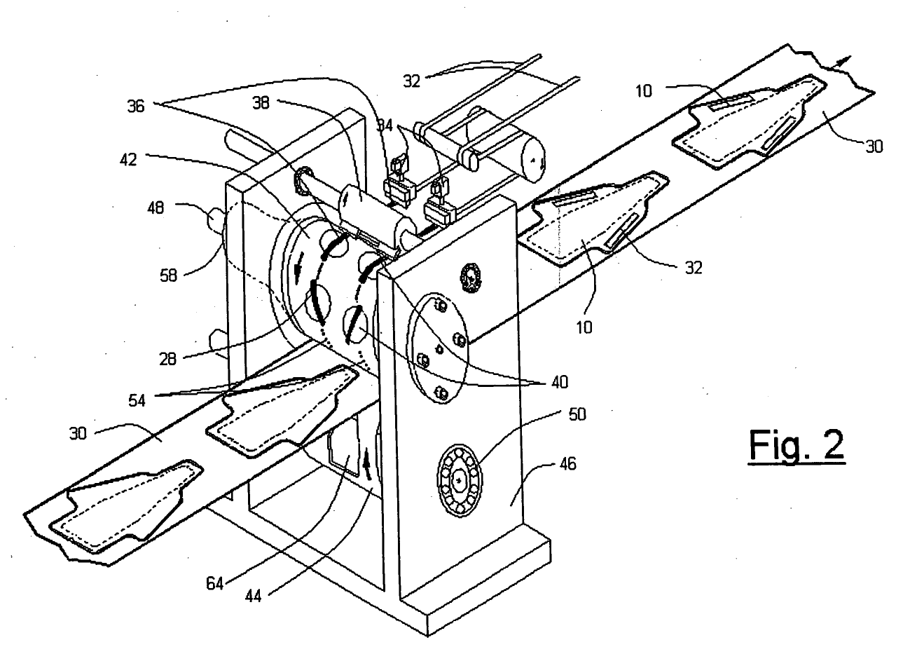

With reference to FIG. 2, the apparatus of the invention is distinctively constructed to more effectively adhere individual adhesive portions 28 to angled, spaced-apart regions of a moving belt 30. Release liner 32 feeds into adhesive applicators 34 that deposit a selected coating of adhesive 36 (e.g., hotmelt adhesive) onto the release liner 32. Regulating means can automatically adjust and maintain a selected cross-directional registry between applicator 34 and release liner 32. A knife roller 38 segments the coated release liner 32 into individual portions 28 on the rotatable disks 40 of an anvil sleeve 42 that provides a selected spatial segregation between the portions 28. The adhesive portions 28 are pressed onto the backsheet layer 12 of a sanitary napkin 10 in the nip between the anvil sleeve 42 and a pressure roller 44.

The various rollers described herein are arranged and rotatably supported within a suitable frame 46, and conventional drive mechanisms (not shown) are operably connected thereto employing ordinary techniques well known to persons skilled in the art. For example, in a particular embodiment of the invention, anvil sleeve 42 is driven by a drive shaft 48 employing conventional power transmission and clutch mechanisms. Roller bearing assemblies 50 can be used to support the rollers in the frame 46.

In a preferred embodiment, applicator 34 deposits a suitable coating of adhesive 36 onto the adjacent surface of release liner 32 in a selected pattern and at a selected add-on amount. In the illustrated embodiment, for example, applicator 34 extrudes a substantially continuous adhesive layer having a generally uniform thickness over the surface of moving release liner 32. The adhesive is preferably a pressure-sensitive adhesive. Suitable pressure sensitive adhesives include for example water-based adhesives such as acrylate adhesives. Alternatively, the adhesive may comprise hot melt adhesives or two-sided adhesive tape. The hot melt adhesives useful for the present invention includes styrenic block copolymers. In a preferred embodiment, the adhesive is based on a high S-I-S (styrene-isoprene-styrene) block copolymer. Suitable adhesives are commercially available from H.B. Fuller Company and ATO Findley. The adhesive may be applied to form the adhesive portion by any means known in the art, such as slot coating, transfer coating, control coating, printing and the like. These applications can result in a continuous coating, a series of strips, a pattern of discrete spots, and the like.

The adhesive applicators 34 may be configured to deposit a regular or irregular pattern of adhesive to provide a continuous or discontinuous coating, as desired, to form the portion. In a particular embodiment of the invention, adhesive applicator 34 is configured to extrude a substantially uniform, continuous coating of adhesive 36 over substantially the entire area of the adjacently located surface of release liner 32. The amount of adhesive add-on is approximately 15 to about 30 g/m².

The adhesive portion 26 may be cooled, e.g., at the anvil sleeve 42 or by separate cooling means, e.g., a chill roller (not shown). The adhesive surface portion 26 is rotated on the disk 40 to an appropriate angle for deposition onto the backsheet layer 12 of the sanitary napkin 10.

Knife roller 38 includes cutting blades 39 that engages and separates a portion 28 of coated release liner 32. The speed of knife roller 38 is suitably coordinated with the speed of belt 30 such that one reinforcement portion 28 is produced for each machine-direction length of belt 30 that corresponds to a single napkin article 10. Once knife roller 38 engages and segments release liner 32 into individual portions 28, the vacuum anvil sleeve 42 forcibly engages the separated portion 28 and accelerates it to provide a selected phasing and spatial segregation between the individual portions. In addition, the acceleration of the segregated portions substantially matches the speed of the portion to the speed of web 30 to allow an accurate adhesive assembly between the portion and the moving napkin 10. To further enhance the adhesion between the portion and the web layer, a rotatable pressure roller 44 engages the laminated arrangement of napkin 10 and portion 28 to provide a pressuring force that enhances the adhesion between the individual portions and the moving napkin 10. Once the portions 28 are secured to napkin 10, the assembly is transported for further processing.

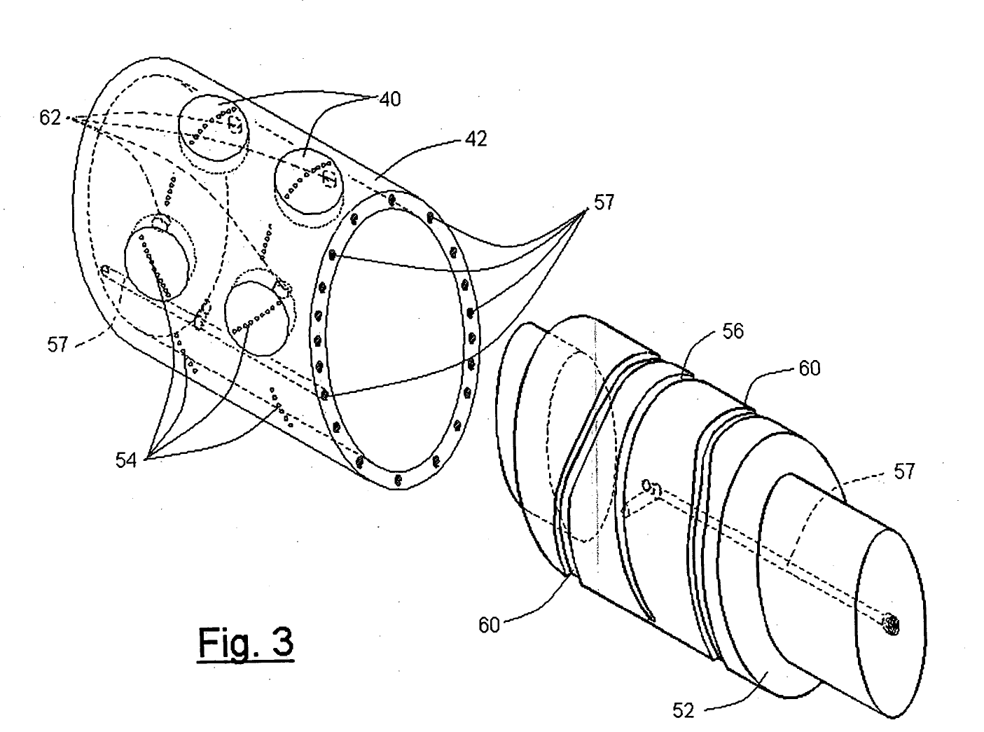

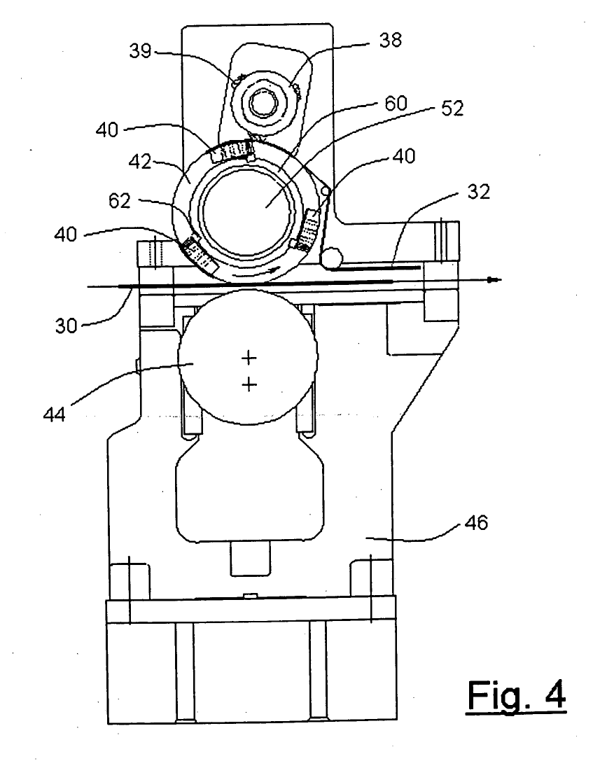

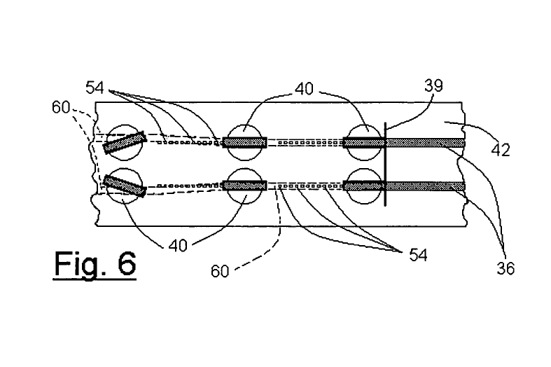

As shown in FIG. 2, anvil sleeve 42 comprises an outer hardened steel shell with bearings. Anvil sleeve 42 is supported on camshaft 52. As shown in FIG. 3, vacuum holes 54 are drilled in a line through the rotatable disks 40 and into a void in fluid communication with a vacuum supply channel 56 and vacuum supply ducts 57. A conventional vacuum manifold 58 is located on at least one axial end of the anvil sleeve 42 in a manner well known in the art to provide a suitable vacuum and is in fluid communication with the vacuum supply channel 56. Blow-off ports are provided on each vacuum manifold 58 to ensure that untransferred substrate portions are reliably removed from the anvil sleeve 42 and directed away from the apparatus. Anvil sleeve 42 is rotated by a suitable drive mechanism (not shown) to produce a speed at its peripheral surface that substantially matches the speed of belt 30.

The camshaft 52 is mounted on the frame 46, and has a central axis. It has at least one fixed cam track 60 formed in the circumferential surface of the camshaft 52. Generally, the number of cam tracks in the camshaft corresponds to the number of attachment elements to be affixed to the absorbent article. As shown in the embodiment of FIGS. 2 and 3, an embodiment of the present invention includes a pair of fixed cam tracks 60 formed in the camshaft 52. The cam followers 62 disposed on the inner surface of each of the rotatable disks 40 engage the fixed cam tracks 60 to direct the rotation of the disks 40. The cam tracks 60 are shown as symmetrical across a plane bisecting the central axis of the cylindrical camshaft 52. This is helpful if the attachment elements 28 are to be symmetrically located on the absorbent article 10. Further, in such an embodiment, pairs of rotatable disks 40 are located on the generatrix (the line which is rotated about the central axis to define the cylinder) of the cylindrical anvil sleeve at a given position, such that the pair moves together on the anvil sleeve 42. It is also useful if the pairs of the rotatable disks 40 are located at equal angular spacing around the surface of the anvil sleeve 42.

The pressure roller 44 is rotatably mounted on the frame 46 in a manner to provide a pressuring force that enhances the adhesion between the individual portions 28 and the moving napkin 10. The pressure roller 44 may be adjustable to allow for adjusting the nip between it and the anvil sleeve 42. The pressure roller 44 may also have a series of pockets 64 to accept the major portion of the napkin 10 to prevent its over-compression in the nip. The surface of the pressure roller 44 may be formed of an elastomeric material to provide resilient backpressure to the anvil roller.

The specification and embodiments above are presented to aid in the complete and non-limiting understanding of the invention disclosed herein. Since many variations and embodiments of the invention can be made without departing from its spirit and scope, the invention resides in the claims hereinafter appended.

1. An absorbent article comprising an absorbent body enclosed between a liquid-permeable liner sheet layer and a liquid-impermeable backsheet layer; wherein the article has a longitudinal axis, a relatively wide anterior end and an opposite posterior end connected by side margins to define a generally triangular shape; the side margins substantially continuously converge to the posterior end; at least one tab extends from each side margin; and at least one attachment element is associated with each tab, the at least one attachment element aligned oblique to the longitudinal axis of the article/having a major axis oriented at an angle that is oblique to the longitudinal direction.

2. The absorbent article of claim 1 wherein the side margins have an inner edge that is substantially straight.

3. The absorbent article of claim 1 wherein only one tab extends from each side margin.

4. The absorbent article of claim 3 wherein only one attachment element is associated with each tab.

5. The absorbent article of claim 4 wherein the attachment elements are affixed to the tabs.

6. The absorbent article of claim 1 wherein each attachment element comprises an adhesive portion.

7. The absorbent article of claim 6 wherein the adhesive portion is protected by a release liner.

8. The absorbent article of claim 1 wherein the article has a thickness of less than about 5 mm.

9. The absorbent article of claim 1 wherein the liner sheet layer and backsheet layer are joined at the side margins.

10. The absorbent article of claim 1 wherein the at least one tab is spaced from the anterior and posterior ends.

11. The absorbent article of claim 10 wherein the at least one tab is centrally located along the side margin.

12-18. (canceled)

19. An absorbent article comprising an absorbent body enclosed between a liquid-permeable liner sheet layer and a liquid-impermeable backsheet layer; wherein the article has a longitudinal axis, a relatively wide anterior end and a posterior end connected by side margins to define a generally triangular shape, the article further comprising at least one tab extending from each side margin, and the article being formed by a process comprising the steps of:
a) separating a first attachment element from a first supply of attachment material that is aligned in a machine direction, parallel to the longitudinal axis of the article;
b) rotating the first attachment element from the machine direction to a predetermined angle that is oblique to the machine direction;
c) indexing the first attachment element to the absorbent article moving in the machine direction; and
d) applying the first attachment element to the at least one tab of absorbent article.

20. The absorbent article of claim 19 formed by a process comprising the further steps of:
e) separating a second attachment element from a second supply of attachment material that is aligned in the machine direction, parallel to the longitudinal axis of the article;
f) rotating the second attachment element from the machine direction to a predetermined angle that is oblique to the machine direction;
g) indexing the second attachment element to the absorbent article moving in the machine direction; and
h) applying the second attachment element to the a second tab of absorbent article.

21. The absorbent article of claim 20 wherein the first and second attachment elements are symmetrical about the longitudinal axis.

22. The absorbent article of claim 19 wherein each attachment element comprises an adhesive portion.

23. The absorbent article of claim 22 wherein the adhesive portion is protected by a release liner.

24. The absorbent article of claim 19 wherein the article has a thickness of less than about 5 mm.

25. The absorbent article of claim 19 wherein the liner sheet layer and backsheet layer are joined at the side margins.

26-37. Cancelled.
